(19) United States
(12) Patent Application Publication
(10) Pub. No.: US 2025/0288257 A1
(43) Pub. Date: Sep. 18, 2025

(54) METHODS AND SYSTEMS FOR VISUALIZING PATIENT STATUS

(71) Applicant: GE Precision Healthcare LLC, Waukesha, WI (US)

(72) Inventors: Virpi Kristiina Lahdenmäki, Uusimaa (FI); Milvi Kristiina Soosalu, Uusimaa (FI)

(21) Appl. No.: 18/608,255

(22) Filed: Mar. 18, 2024

(51) Int. Cl. A61B 5/00; G16H 10/60; G16H 50/30

(52) U.S. Cl. CPC A61B 5/743; A61B 5/7435; A61B 5/7445; G16H 10/60; G16H 50/30

(57) ABSTRACT

A system is disclosed for visualizing physiological patient data collected in real time from a patient monitor and historical physiological patient data retrieved from a database, in a single display. The system generates and displays a patient status visualization including a radar chart that summarizes patient data according to different systems of the patient, with a possibility to view detailed parameter data specific to each system, facilitating an efficient and timely process for determining patient status. A set of chronologically organized historical radar charts similar to the radar chart display of the real-time patient data may be generated from the historical patient data. The chronologically organized historical radar charts may be displayed via a slider element, where a user may view a chronological progression of the historical radar charts by sliding a slider element of the patient status visualization, ending with the radar chart showing the real-time patient data.

### FIELD

Embodiments of the subject matter disclosed herein relate to summarizing and displaying clinical patient data.

### BACKGROUND

Monitoring physiological patient data of a patient is an important part of patient care, and physicians often desire to continuously monitor multiple physiological patient data of their patients in real time, such as blood pressure, oxygen saturation (SpO2), features of the electrocardiogram (ECG), and others. Patient monitoring often involves the use of several sensing devices to perform multiple physiological monitoring modalities, such as a pulse oximeter, a blood pressure monitor, a heart monitor, a temperature monitor, etc. Many patient monitoring devices offer multi-modality patient monitoring, where multiple different sensing devices for sensing different physiological patient data can be connected to a single patient monitor that is configured to collect, process, and/or display physiological information describing the patient's health condition. The patient monitor may also be connected to wireless network accessible in the hospital environment, such that the multiple different sensing devices may communicate with the patient monitor wirelessly. A caregiver may monitor real-time physiological patient data of the patient remotely by viewing data of the patient monitor via a remote viewing application (e.g., on a smart phone), as the patient or the caregiver move around a hospital environment.

However, reviewing the real-time physiological patient data to determine a status of a patient may be cumbersome and time consuming. A number of clinical parameters acquired from a patient may be high, and the parameter information may be presented individually and separately from each other. Additionally, a screen of a patient monitor or smart phone on which the real-time physiological patient data is displayed may be small, such that the caregiver may have to scroll or navigate through various screens of data. As a result, the caregiver may have to rely on combining the data and drawing appropriate conclusions regarding the patient status in their head. This may increase cognitive overload, which may result in inaccurate assessments of patient status.

This problem may be exacerbated when the caregiver wishes to consult historical data to compare with the real-time physiological patient data received from the patient monitor. The historical data may be stored in an Electronic Health Record (EMR) system, where sorting and extracting information may be a slow and inefficient process, and determining the status of a patient may include remembering patient data accessed from a variety of sources. As a result, determining and monitoring patient statuses may consume a large proportion of the caregiver's time, consequently reducing an amount of time available for tending to patients. Additionally, inefficiencies and risks of incorrectly estimating patient statuses may increase a likelihood of poor patient outcomes.

### BRIEF DESCRIPTION

The patent or application file contains at least one drawing executed in color. Copies of this patent or patent application publication with color drawings will be provided by the Office upon request and payment of the necessary fee.

In one example, the problems described above are addressed by a method for a patient status visualization system, the method comprising receiving physiological patient data generated by one or more sensors arranged on a patient, in real time; retrieving historical physiological patient data corresponding to the physiological patient data from one or more systems and/or databases communicatively coupled to the patient status visualization system, and storing the retrieved historical physiological patient data in a memory of the patient status visualization system; generating a patient status visualization of a status of the patient, based on the real-time physiological patient data; displaying the patient status visualization on a display device; and in response to a user adjusting a control element of the patient status visualization, retrieving one or more elements of the historical physiological patient data from the memory and displaying the one or more elements in the patient status visualization in real time as the user adjusts the control element.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will be better understood from reading the following description of non-limiting embodiments, with reference to the attached drawings, wherein below:

FIG. 1 illustrates an exemplary patient monitoring environment;

FIG. 2 is a schematic diagram showing an exemplary patient status visualization system, which may automatically generate patient status visualizations for a caregiver;

FIG. 3A shows a first exemplary patient status visualization displayed by the patient status visualization system;

FIG. 3B shows a second exemplary patient status visualization displayed by the patient status visualization system;

FIGS. 4A and 4B are flowcharts that illustrate exemplary methods for generating patient status visualizations from physiological patient data;

FIG. 5 shows a third exemplary patient status visualization displayed by the patient status visualization system;

FIG. 6 shows a first pop-up display panel including an exemplary patient status visualization displayed by the patient status visualization system;

FIG. 7 shows a second pop-up display panel including an exemplary patient status visualization displayed by the patient status visualization system; and

FIG. 8 shows a fourth exemplary patient status visualization displayed by the patient status visualization system.

### DETAILED DESCRIPTION

The following description relates to systems and methods for quickly and efficiently assessing a status of patients in care units of a hospital or healthcare organization. Efficient patient management may include stabilizing patients with serious medical conditions and providing initial treatment, and once the patient is stable, diagnosing the patient and establishing a treatment strategy for the patient. Thus, efficient patient management may depend on an efficient patient monitoring process for regularly assessing a status of a plurality of patients.

Patient monitoring may include a number of different physiological monitoring devices, sensors, etc. capable of monitoring cardiac, respiratory, neurologic, hemodynamic, pulse oximetry, etc. parameters such as but not limited to electrocardiogram (ECG), peripheral capillary oxygen saturation (SpO2), respiration rate, temperature, blood pressures, Entropy, blood glucose, and carbon dioxide. Patient monitoring is performed by way of many different forms and approaches with respect to data capture and communication technologies (e.g., hard-wired and wireless networking) and may include monitoring a patient locally (e.g., in-room wired or tethered to a monitor) and/or wirelessly (e.g., in-room, while in transport, ambulating telemetry). In addition to moveable roll stand and room-based semi-fixed or permanently mounted physiological patient data acquisition equipment acquiring one or more parameters, monitoring may be performed with small, portable devices (whether as multiple separate sensors or as an integrated acquisition device) coupled to the patient in order to enable the patient to ambulate (e.g., walk) remotely relative to a designated hospital bed or treatment room while maintaining monitoring of the condition of the patient (e.g., heart rhythm, oxygenation, and other patient vital signs). For example, ambulation of the patient may be desirable for resolution of various medical conditions for which the patient is being treated (e.g., chest pain, syncope, post-surgical). A caregiver (e.g., nurse, doctor, or another clinician) may view an output of the monitoring device(s) on the device's user interface, at a remote location such as a patient monitoring central station, via another system such as an Electronic Medical Record (EMR) system, or at a handheld device such as a smart phone or tablet, throughout the duration that the monitoring device(s) is attached or coupled to the patient.

In various situations, the caregiver may wish to consult historical physiological patient data to compare with the real-time physiological patient data. For example, the caregiver may wish to compare a current blood pressure with a historical blood pressure of the patient at different times in the past. The physiological patient data to be reviewed may be stored in an Electronic Medical Record (EMR) system, which may comprise, or aggregate physiological data from a plurality of different computer systems and databases, for example, via a hospital network. Sorting through and extracting the physiological patient data may be a slow and inefficient process. For example, to retrieve a specific physiological parameter (e.g., blood pressure) at a specific time, the caregiver may have to navigate through various menus and screens of the EMR, select, define, and/or configure various monitor trend views, etc.

Accordingly, when the caregiver evaluates a current status of the patient in light of the historical physiological data, the caregiver may undergo a time-consuming and mentally burdensome process where the caregiver manually selects records of the EMR or parameter data stored in the monitoring system to review. For the caregiver, figuring out the patient's status, how the patient is responding to treatment, and a relationship of the patient's historical physiological data to the current patient status may place a large cognitive load on the user, increasing an amount of time the caregiver spends consulting the patient data and reducing the number of patients the caregiver may attend to. Without any mechanism to help the user quickly hone in on relevant information, information may be missed that may result in an inappropriate assessment of the patient's status, compromising patient care. Further, the existing system forces the user to navigate through individual data elements, parameters, and/or medical records, which may result in the user selecting and reviewing data that is not relevant, selecting unwanted data in error, etc., which may make an underlying computing system itself inefficient because the computing system may be asked to retrieve and display more information than necessary.

Thus, according to embodiments disclosed herein, a patient status visualization system may be employed to retrieve and compile relevant patient status information from a variety of sources, including real-time data from patient monitors and historical data stored in clinical databases, and automatically generate and display a patient status summary visualization of the patient's status. The patient status summary visualization includes a radar chart that summarizes and presents a limited amount of patient data relevant to the patient's status at a high level, thereby facilitating an efficient and timely decision-making process for determining a current status of the patient. The generation and display of the radar chart may reduce a footprint of the patient medical data and may reduce the cognitive load on the user/clinician, while highlighting the major health issues affecting patients. The radar chart summarizes patient status per organ or system of the patient, and provides a possibility to drill down to view detailed clinical parameter data specific to that organ or system, or additional visualizations. In this way, complex real-time information can be combined together to provide new insights, such as changes in a hemodynamic status of the patient from various clinical data, that might be challenging to do by extracting relevant numbers from a display.

In particular, the patient status visualization system offers a novel approach to integrating the visualization of real-time patient data with historical patient data. Historical patient data matching the real-time patient data displayed in the patient status summary visualization may be retrieved from one or more clinical information systems and/or databases, and processed to generate a set of chronologically organized historical radar charts similar to the radar chart display of the real-time patient data. The chronologically organized historical radar charts may be displayed via a slider element of the patient status summary visualization. A user may view a chronological progression of the historical radar charts by sliding the slider element, where the chronological progression may end with the radar chart showing the real-time patient data. In this way, current patient data may be quickly and efficiently compared to historical patient data in a user-friendly manner.

Further, differences between the historical patient data and the real-time patient data may be quickly identified based on deviations in a shape of the radar charts over time, as the user adjusts the slider element. In this way, an efficiency of the underlying computing system is increased, by advantageously retrieving, processing, and reformatting the historical patient data for efficient display at a first time, such that the chronological progression of the historical patient data may be displayed as an interactive animation in real-time (e.g., with no intentional delay) in response to the user adjusting the slider element at a second, later time. Such an animation would not be possible in either an alternative system where elements of the historical patient data were retrieved for display via individual queries submitted in real time, or an alternative system where the historical patient data was not reformatted into the patient status summary visualization as described above.

Thus, the automatic generation and display of patient status visualizations may reduce an amount of time spent by a caregiver in determining statuses of a rotating body of patients, thereby increasing an amount of time available to the caregiver to attend to patients, and allow a greater number of criteria to be considered in making a patient status visualization. Further, a cognitive load placed on the caregiver may also be reduced, thereby reducing a risk of error in assessing patient status.

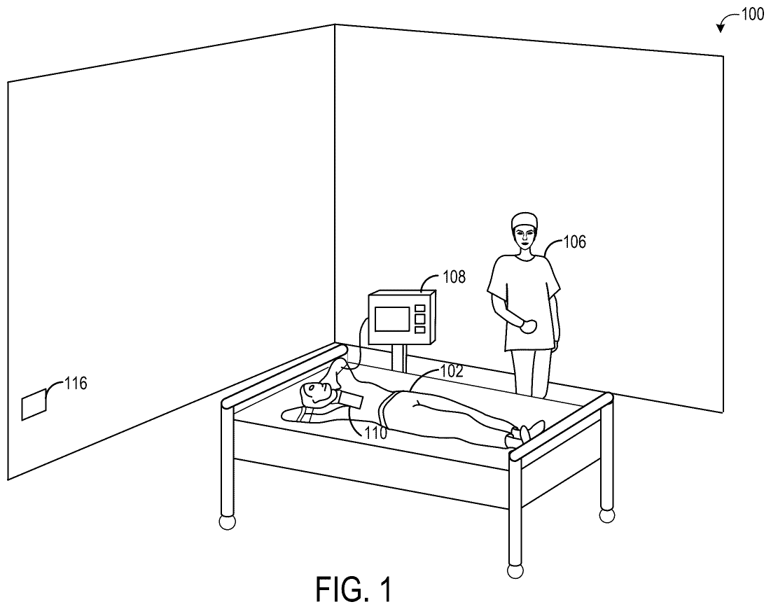

Embodiments of the present disclosure will now be described, by way of example, with reference to the figures. Referring now to FIG. 1, it shows an example patient monitoring environment 100, which in the depicted example is a patient room in a hospital or other medical facility. The patient monitoring environment 100 may include one or more patient monitoring devices or patient data acquisition devices that monitor or acquire one or more physiological patient data, as well as treatment devices. The monitoring environment 100 includes a patient 102 being monitored by a plurality of monitoring devices and also being attended to by a clinician 106. Clinician 106 may be a nurse, physician, medical technologist, or another suitable medical professional. The monitoring devices include a bedside device 108 and a patient monitor 110. In some embodiments, either or both of bedside device 108 and patient monitor 110 may be patient data acquisition devices. The monitoring environment 100 depicts the use of more than one monitoring device but it is understood this is non-limiting as the environment 100 could include one device monitoring one parameter, one device monitoring more than one parameter, multiple devices each monitoring one or more than one parameter, and so on. Due to differing patient conditions and the varying patient monitoring needs, one or more devices may be used to support the monitoring needs of the patient and capable of supporting monitoring in various conditions (e.g., in room, in transport, patient ambulation). The one or more bedside devices 108 may be included or mounted in a floor, table top, or roll stand module that includes one or more leads or other components coupled to the patient or in wireless communication to a device connected to the patient, in order to monitor one or more parameters of the patient (such as ECG, respiration, blood pressure, carbon dioxide levels, etc.). The one or more bedside devices 108 are configured to remain in the patient room, and thus patient 102 may have limited mobility when coupled to the one or more bedside devices 108.

Patient monitor 110 may include one or more telemetry devices (with different sensor capabilities) housed in a common module (as shown) or housed in two or more separate modules. Patient monitor 110 may be positioned on the patient (e.g., via a pouch, holder, or similar, attached to a belt of the patient) or on a movable module (e.g., a wheeled module), such that patient monitor 110 may leave the patient room if the patient leaves the patient room, and may travel with the patient. Patient monitor 110 may be connected to the patient 102 via one or more leads or other components or in wireless communication with an associated sensor, in order to monitor one or more parameters of the patient (such as ECG, respiration, blood oxygen level, etc.).

The patient monitoring data collected by patient monitor 110 may be sent wirelessly (e.g., WiFi, Bluetooth, MBAN) and/or via a hard-wired networked connection to one or more associated devices for processing, analysis, storage, display, etc., such as a central station, patient monitoring database, and/or different patient monitoring system. The methods of wireless communication used by patient monitor 110 and the receiving systems vary widely based on the technology used. In one example communication approach, to facilitate the transfer of the patient monitoring data collected by patient monitor 110, patient monitoring environment 100 and nearby areas (e.g., hallways, closets, open spaces) may include one or more access points 116. Access point 116 may receive and send information (e.g., wirelessly) to patient monitor 110 (e.g., the patient monitoring data, communication status). The access point 116 sends the received information to a processing server, a central station, a telemetry monitoring system, and/or another suitable device. If patient 102 leaves the patient room and moves throughout the medical facility, patient monitoring data collected by patient monitor 110 may be sent to other access points located throughout the medical facility. Patient monitoring data collected by the one or more bedside devices 108 may likewise be sent to a processing and analysis server, the central station, the telemetry monitoring system, and/or another suitable device, via wireless communication with access point 116 or another access point, or via a wired connection. It is understood, this example using a transceiver and access point for data communications is one of many different technologies suitable for sharing acquired patient monitoring data with the associated data processing, analysis, storage, and information viewing system components and infrastructure.

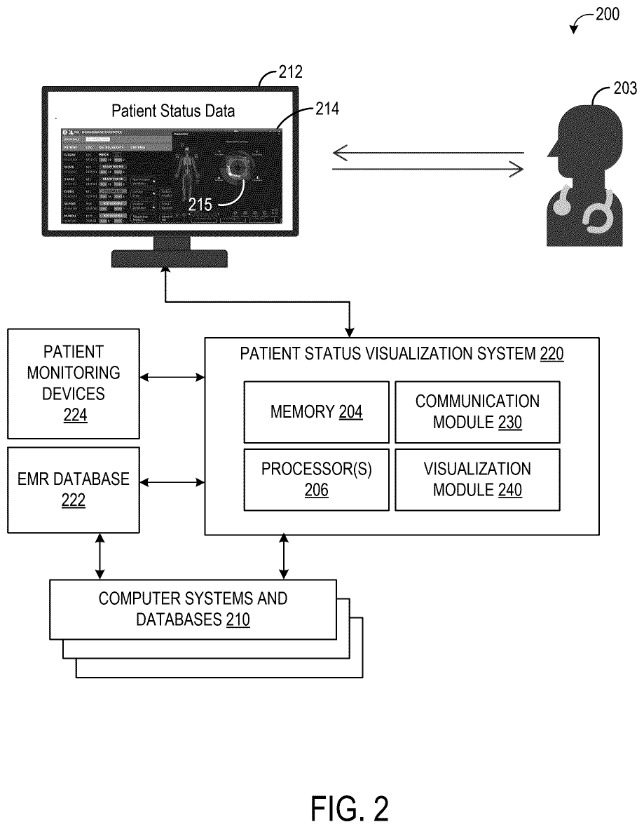

FIG. 2 shows a clinical information system 200 according to an aspect of the disclosure. The clinical information system 200 may include a patient status visualization system 220 configured for generating patient status visualizations and associated clinical information of a patient that may be displayed to a user 203 via a display 212. The display 212 may be any known device having a screen to display the patient status visualizations and associated clinical information. The display device 212, and in some examples, more than one display device, may be communicatively coupled to patient status visualization system 220.

The display device 212 may include a processor, memory, communication module, user input device, display (e.g., screen or monitor), and/or other subsystems and may be in the form of a desktop computing device, a laptop computing device, a tablet, a smart phone, or other device. The display device 212 may be adapted to send and receive encrypted data and display medical information, including medical images in a suitable format such as digital imaging and communications in medicine (DICOM) or other standards. The display device 212 may be located locally at a medical facility (such as in a care unit of the medical facility) and/or remotely from the medical facility (such as a caregiver's mobile device). In some embodiments, the display device 212 may be a non-limiting example of patient monitor 110 and/or bedside device 108.

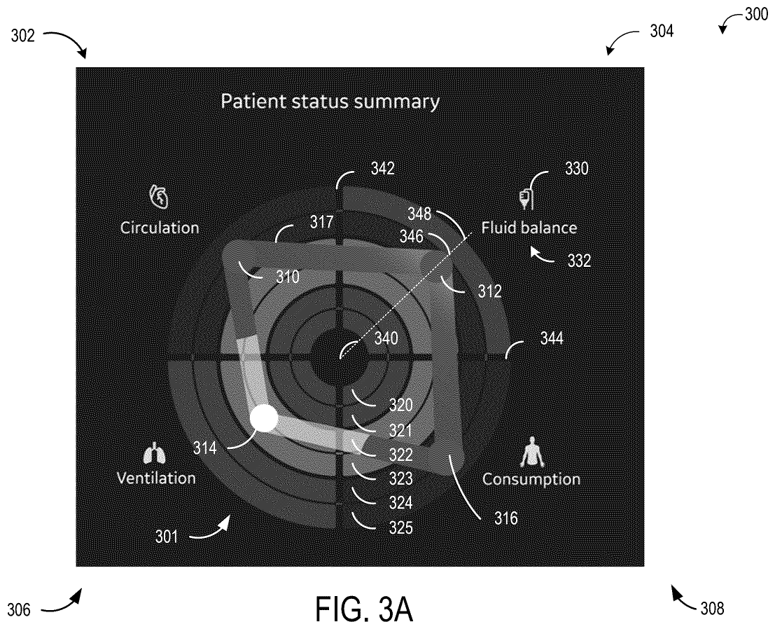

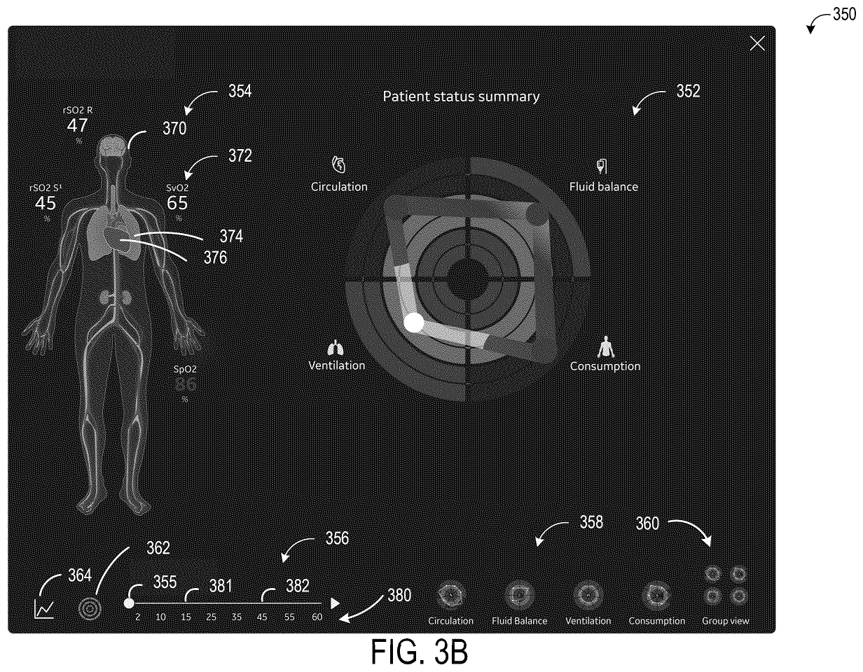

The patient status visualizations may be viewed via a user interface (UI) 214 (which may be a graphical user interface and thus also referred to as a GUI) of the display device 212. The patient status visualizations may comprise one or more graphical elements 215. In particular, the graphical elements 215 may include a radar chart summarizing the status of the patient, as described in greater detail below in reference to FIG. 3A. When viewing the patient status visualizations via the UI 214, the user may enter input (e.g., via a user input device, which may include a keyboard, mouse, microphone, touch screen, stylus, or other device) that may be processed by the patient status visualization system 220. In examples where the user input is a selection of a link or control button of the UI 214, the user input may trigger display of additional clinical information relevant to a patient status, or other actions.

The clinical information system 200 may include an EMR database 222, which may be communicatively coupled to the patient status visualization system 220. EMR database 222 may be stored in a mass storage device configured to communicate with secure channels (e.g., HTTPS and TLS), and store data in encrypted form. Further, the EMR database/computing system may be configured to control access to patient electronic medical records such that only authorized caregivers may edit and access the electronic health records. An EMR for a patient may include medical device data, including historical patient monitoring data (e.g., vital signs measured or otherwise ascertained by medical devices such as heart rate, oxygen saturation, etc.), user-specified medical parameters (e.g., acuity scores, pain scores), patient demographic information, family medical history, past medical history, lifestyle information, preexisting medical conditions, current medications, allergies, surgical history, past medical screenings and procedures, past hospitalizations and visits, etc.

In addition to the EMR database 222, historical patient information that is integrated into the patient status visualizations may also be stored in one or more computer systems and databases 210 in communication with patient status visualization system 220 and/or the EMR database 222. For example, the computer systems and databases 210 may include a picture archiving and communication system (PACS) that may store and communicate medical images and associated reports (e.g., clinician findings), such as ultrasound images, MRI images, etc., according to the DICOM format. The computer systems and databases 210 may include a radiology information system (RIS), which may store radiology images and associated reports, such as CT images, X-ray images, etc. The computer systems and databases 210 may include a laboratory database and/or a pathology database, which may store lab results, pathology images and related reports, including visible light or fluorescence images of tissue, such as immunohistochemistry (IHC) images. It should be appreciated that the example computer systems and databases provided herein are for illustrative purposes, and in various embodiments, additional, fewer, or different computer systems and/or databases may be included without departing from the scope of this disclosure.

Patient status visualization system 220 may include a memory 204 and a processor(s) 206 to store and execute the methods disclosed herein to generate the patient status visualizations and patient status data, as well as send and receive communications, graphical user interfaces, medical data, and other information. Memory 204 may include one or more data storage structures, such as optical memory devices, magnetic memory devices, or solid-state memory devices, for storing programs and routines executed by processor(s) 206 to carry out various functionalities disclosed herein. Memory 204 may include any desired type of volatile and/or non-volatile memory such as, for example, static random access memory (SRAM), dynamic random access memory (DRAM), flash memory, read-only memory (ROM), etc. Processor(s) 206 may be any suitable processor, processing unit, or microprocessor, for example. Processor(s) 206 may be a multi-processor system, and, thus, may include one or more additional processors that are identical or similar to each other and that are communicatively coupled via an interconnection bus.

In some examples, patient status visualization system 220 may be implemented over a cloud or other computer network. For example, patient status visualization system 220 is shown in FIG. 2 as constituting a single entity, but it should be appreciated that patient status visualization system 220 may be distributed across multiple devices, such as across multiple servers.

Patient status visualization system 220 may be configured to generate and output one or more patient status visualizations for display via display 212. Generating the patient status visualizations may reduce a time spent by the user reading information for a current patient that is not relevant to a patient status, allowing the user to view information for a plurality of patients with increased efficiency. Each patient status visualization may include real-time patient monitoring data including clinical markers (also referred to as criteria) relevant to the patient status visualization, as well as historical physiological patient data related to the real-time patient monitoring data. The historical physiological patient data may be automatically extracted from the EMRs and/or other medical information of the patient (e.g., pathology reports, imaging scans/reports, etc.) that may or may not be stored in the EMRs when the patient status visualization is first launched and/or configured.

For example, the real-time patient monitoring data may include a current blood pressure and heart rate of a patient, and the historical physiological patient data may include prior blood pressure and heartrate measurements of the patient made at previous times. The real-time patient monitoring data and the historical physiological patient data may be integrated into a patient status visualization, such that a clinician may easily compare the real-time patient monitoring data with corresponding historical physiological patient data. In particular, as described in greater detail below, graphical element 215 (e.g., the radar chart) may be interactive, where the clinician may view both a first radar chart showing the real-time patient monitoring data, and a second, similarly configured radar chart showing the historical physiological patient data. That is, the clinician may toggle between the first radar chart and the second radar chart by adjusting a control element in GUI 214, or view a progression of physiological patient data from the historical patient data to the (current) real-time patient monitoring data by adjusting a slider or similar control element of GUI 214. In some examples, elements of the historical physiological patient data and/or real-time patient monitoring data may be assembled into a natural-language-like format and displayed along with and/or adjacent to a relevant patient status visualization.

Further, the historical physiological patient data may be integrated into patient status visualization at a time when the patient status visualization is configured by the clinician, rather than at a later time when the clinician wishes to compare the real-time patient monitoring data with the historical physiological patient data, such that the historical physiological patient data may not be retrieved on demand from an EMR. As a result, a responsiveness of GUI 214 and graphical element 215 to the user interaction may be increased, such that the patient status visualization may be quickly adjusted as the clinician adjusts a relevant control element.

Thus, an additional advantage of the patient status visualization system is that historical patient data may be viewed and quickly compared with real-time patient monitoring data even while one or more EMR systems are in an unlaunched state (e.g., not consuming computing and/or network resources of the healthcare system), or when the EMR database may not be accessible via a hospital network. That is, an EMR system accessed by the patient status visualization system may become temporarily or periodically unavailable, for example, if a system or network coupled to the patient status visualization system experiences a failure.

As used herein, the term “module” may include a hardware and/or software system that operates to perform one or more functions. For example, a module may include a computer processor, controller, or other logic-based device that performs operations based on instructions stored on a tangible and non-transitory computer readable storage medium, such as a computer memory. Alternatively, a module may include a hard-wired device that performs operations based on hard-wired logic of the device. Various modules shown in the attached figures may represent the hardware that operates based on software or hardwired instructions, the software that directs hardware to perform the operations, or a combination thereof.

“Modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

Patient status visualization system 220 may include a communication module 230. Communication module 230 may facilitate transmission of electronic data within and/or among one or more systems. Communication via communication module 230 can be implemented using one or more protocols. In some examples, communication via communication module 230 occurs according to one or more standards (e.g., Digital Imaging and Communications in Medicine (DICOM), Health Level Seven (HL7), ANSI X12N, etc.). Communication module 230 can be a wired interface (e.g., a data bus, a Universal Serial Bus (USB) connection, etc.) and/or a wireless interface (e.g., radio frequency, infrared, near field communication (NFC), etc.). For example, communication module 230 may communicate via wired local area network (LAN), wireless LAN, wide area network (WAN), etc. using any past, present, or future communication protocol (e.g., BLUETOOTH™, USB 2.0, USB 3.0, etc.).

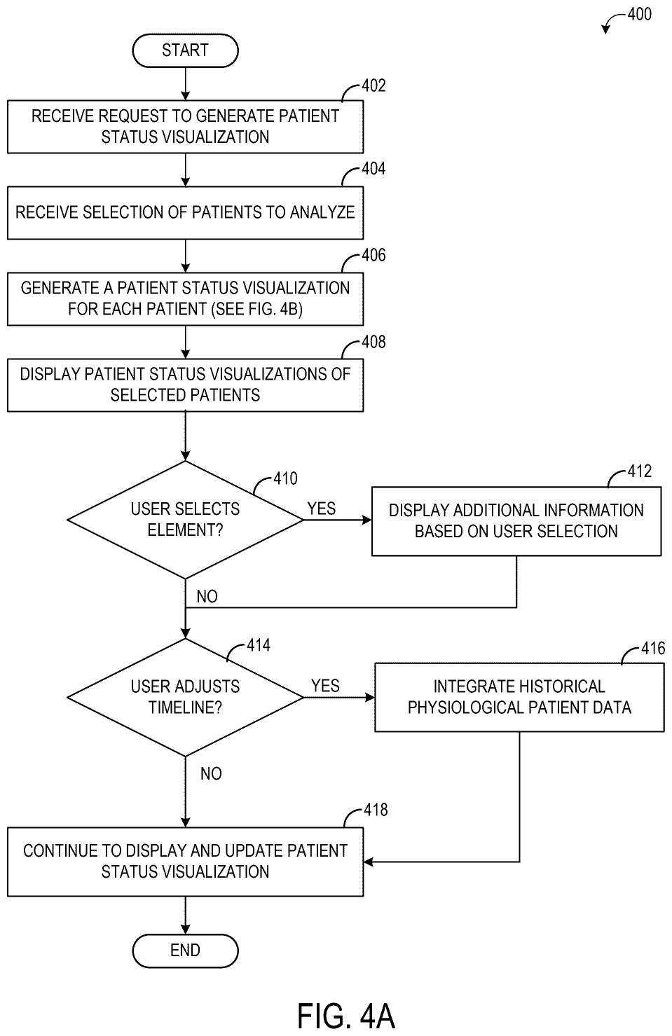

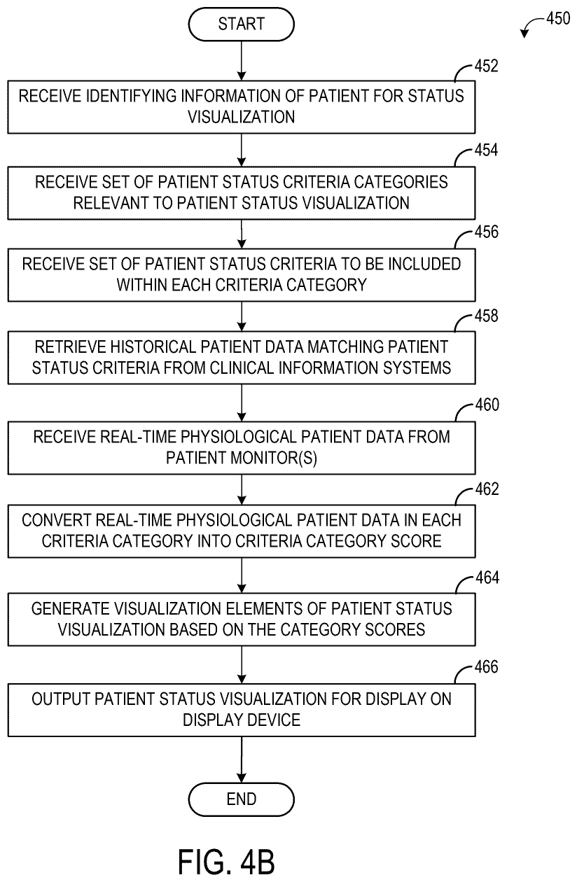

Patient status visualization system 220 may include a visualization module 240. The visualization module 240 may store one or more models that may be used to generate the patient status visualizations. The one or more models may include a rules-based system, where machine-implementable rules may be applied to a set of patient data relating to a current or prior status of the patient retrieved by the patient status visualization system 220. The one or more models may also include machine learning (ML) or deep learning (DL) models trained to output an indicator of patient status, based on various input data. The generation of the patient status visualizations from the set of patient data is described below, in reference to FIGS. 4A and 4B.

While not specifically shown in FIG. 2, additional devices described herein (e.g., display device 212) may likewise include user input devices, memory, processors, and communication modules/interfaces similar to communication module 230, memory 204, and processor(s) 206 described above, and thus the description of communication module 230, memory 204, and processor(s) 206 likewise applies to the other devices described herein. As an example, the display device 212 may store user interface templates in memory that include placeholders for relevant information stored on patient status visualization system 220 or received via patient status visualization system 220, which a user of display device 212 may configure. The patient status visualizations and relevant patient information may be retrieved from patient status visualization system 220 and inserted in the placeholders. The user input devices may include keyboards, mice, touch screens, microphones, or other suitable devices.

In various examples, the caregiver may open the patient status visualization system by first logging into a patient management system of a healthcare network, and selecting a patient status visualization system icon, tile, or similar menu listing of one or more options for retrieving patient data of the patient management system to start the patient status visualization system. In other examples, the patient status visualization system may be launched from within a different computer system of a hospital, such as an EMR system, where the patient status visualization system may be listed as a selectable menu option within a UI of the EMR system. In general, the patient status visualization system may be integrated into any suitable existing care device of a healthcare or hospital system.

In some embodiments, patient status visualization system 220 may be launched by a caregiver when the caregiver wishes to assess the status of one or more patients. In other embodiments, the patient status visualization system may be continuously running, and elements of the patient status visualization system and/or patient data may be periodically updated automatically by the patient status visualization system. For example, the caregiver may start the patient status visualization system and configure a patient status visualization of a patient at a first time. When the caregiver configures the patient status visualization, the caregiver may specify a set of criteria to be displayed in the patient status visualization. For example, the set of criteria may include various vital signs and/or sensor readings via a patient monitor. The set of criteria may be combined and/or organized into categories, such as, for example, organ specific categories. The patient status visualization may display real-time patient physiological data corresponding to the set of criteria, for example, as a radar chart. Additionally, when the set of criteria is specified, patient status visualization system 220 may retrieve historical patient physiological data corresponding to the set of criteria, for comparative purposes. The historical patient physiological data may be integrated into the patient status visualization as a series of chronologically organized historical radar charts, where the historical radar charts share a same visual structure, configuration, and criteria as the radar chart including the real-time patient physiological data. Thus, the caregiver may select whether to view the radar chart including the real-time patient physiological data, or to view one or more historical radar charts including the historical patient physiological data for the same set of criteria.

The caregiver may leave the patient status visualization system to attend to patients, and return later to the patient status visualization system, which may still be running, to view the patient status visualization of the patient at a second time. In between the first time and the second time, elements of patient data and/or patient status visualizations may change, for example, due to a worsening condition of a patient. Thus, the patient status visualization system may serve to provide continuously updated data that may aid the caregiver in managing patients.

Turning to FIG. 3A, an example of a radar chart 300 of a patient status visualization system is shown, which may be, include, or form a part of graphical element 215 of UI 214 of the patient status visualization system 220 of FIG. 2. Radar chart 300 may be displayed on a display device (e.g., display device 212) of the patient status visualization system. Specifically, radar chart 300 may be displayed to a caregiver when the caregiver uses the patient status visualization system to visualize a status of a patient of the caregiver, based on physiological patient data collected in real time via a patient monitor.

It should be appreciated that the layout and juxtaposition of the elements of radar chart 300 and other visualizations included in this disclosure may vary in different embodiments, and the elements may appear in different visual configurations, including configurations that are configurable by the caregiver or a different user, without departing from the scope of this disclosure. Additionally, not all of the elements shown in radar chart 300 and other visualizations may be included in an embodiment, and some embodiments may include a greater or lesser number of elements.

Radar chart 300 may summarize a status of a patient with respect to different configurable categories of criteria that relate to a status of the patient, where the different categories are represented as conical sections of a circular graphical element 301 arranged around a center point 340. Each criteria category may correspond to a body system of the patient, or an organ system of the patient, or a different meaningful grouping of physiological patient data. In the depicted embodiment, radar chart 300 includes four criteria categories, corresponding to four quadrants of radar chart 300, where the four quadrants are separated by a vertical centerline 342 and a horizontal centerline 344 that intersect center point 340.

Specifically, a first criteria category 302 appearing in an upper left quadrant of radar chart 300 corresponds to patient status criteria associated with a circulatory system of the patient. First criteria category 302 may include patient status criteria such as, for example, a heart rate of the patient, a blood pressure of the patient, oxygen saturation of blood etc. A second criteria category 304 appearing in an upper right quadrant of radar chart 300 corresponds to patient status criteria associated with a balance of an input and output of fluids of the patient. Second criteria category 304 may include patient status criteria such as, for example, a urine output of the patient, a pulse pressure variation (PPV) of the patient, and/or other useful predictors of fluid balance, fluid depletion, fluid responsiveness, etc. A third criteria category 306 appearing in a lower left quadrant of radar chart 300 corresponds to patient status criteria associated with a ventilation or respiratory system of the patient. Third criteria category 306 may include patient status criteria such as, for example, a respiratory rate (RR) of the patient, respiratory minute volume (MV) of the patient, a fraction of inspired oxygen (FiO2), and so on. A fourth criteria category 308 appearing in a lower right quadrant of radar chart 300 corresponds to patient status criteria associated with an oxygen consumption of the patient. Fourth criteria category 308 may include patient status criteria such as, for example, a temperature of the patient, a validation of carbon dioxide production (VCO2) of the patient, a maximum amount of oxygen the patient uses during heavy exercise (VO2), an energy expenditure (EE) of the patient, an arterial-venous oxygen content difference C(a-v)O2 of the patient, a respiratory ratio (RQ) of the patient, and/or other criteria related to oxygen consumption. It should be appreciated that the criteria categories included in FIG. 3A are for illustrative purposes, and in other embodiments, radar chart 300 may include a greater or lesser number of criteria categories, or different criteria categories, without departing from the scope of this disclosure.

Each quadrant of radar chart 300 may include an icon 330 representing the criteria category of the quadrant, and an identifier (e.g., a name of the criteria category). For each of the criteria categories, the patient status criteria included within the criteria category may be summarized by a single category score or value, which may be represented by a point within a corresponding quadrant. Thus, first criteria category 302 may include a first circulatory score represented by a point 310; second criteria category 304 may include a second fluid balance score represented by a point 312; third criteria category 306 may include a third ventilation score represented by a point 314; and fourth criteria category 308 may include a fourth oxygen consumption score represented by a point 316. The generation of the summary scores from the individual patient status criteria values is described below in reference to FIGS. 4A and 4B.

In the depicted embodiment, the points 310, 312, 314, and 316 are positioned along a central axis of a corresponding quadrant, such as the central axis of second criteria category 304, which is indicated by a dotted line 346. In other embodiments, one or more points 310, 312, 314, and 316 may not be positioned along the central axis. A position of a relevant point along the central axis between center point 340 and an outer perimeter 348 of circular graphical element 301 may depend on the score summarizing the patient status criteria of the relevant criteria category. In other words, the positioning of the points 310, 312, 314, and 316 between center point 340 and an outer perimeter 348 establishes a metric for each criteria category, by which a respective score may be measured in relation to an expected range of values of the score and/or minimum and maximum thresholds for the score.

For example, point 312 may be positioned along dotted line 346 based on the second fluid balance score. If the second fluid balance score were to increase, point 312 may be displayed closer to outer perimeter 348, and if the second fluid balance score were to decrease, point 312 may be displayed closer to center point 340. Thus, a summary of the status of the patient with respect to the second criteria category (e.g., fluid balance) may be quickly ascertained by considering the distance between point 312 and center point 340. Similarly, a summary of the status of the patient with respect to the first criteria category (e.g., circulation) may be quickly ascertained by considering the distance between point 310 and center point 340; a summary of the status of the patient with respect to the third criteria category (e.g., ventilation) may be quickly ascertained by considering the distance between point 314 and center point 340; and a summary of the status of the patient with respect to the fourth criteria category (e.g., consumption) may be quickly ascertained by considering the distance between point 316 and center point 340. In this way, an overall status of the patient may be indicated by a relative positioning of points 310, 312, 314, and 316 within radar chart 300. The relative positioning of the points may be further accentuated by drawing a series of lines 317 that connect points 310, 312, 314, and 316, forming a network with an identifiable shape. The identifiable shape may be used to compare different radar charts, such as, for example, radar charts of different patients, or radar charts of a same patient generated with data acquired at different times.

Further, circular graphical element 301 may include a plurality of concentric rings surrounding center point 340, including a first ring 320, a second ring 321, a third ring 322, a fourth ring 323, a fifth ring 324, and a sixth ring 325 at outer perimeter 348. Each ring may represents a degree to which a score represented by a point is within a desired or expected range for the score. For example, point 314 is positioned between third ring 322 and fourth ring 323 (e.g., approximately mid-way between center point 340 and outer perimeter 348), which may indicate that the third ventilation score is at a mid-point of a range of values of the third ventilation score. From the position of point 314, it may be inferred that the values of the patient status criteria included in the third criteria category 306 (e.g., respiratory rate, etc.) may be generally within normal ranges.

Alternatively, point 316 is positioned between fifth ring 324 and sixth ring 325, close to outer perimeter 348, which may indicate that the fourth consumption score is at an upper portion of a range of values of the fourth consumption score. From the position of point 316, it may be inferred that the values of one or more of the patient status criteria included in the fourth criteria category 308 may be higher than expected. If point 316 were positioned close to center point 340, for example, in first ring 320 or second ring 322, it may be inferred that the values of one or more of the patient status criteria included in the fourth criteria category 308 may be lower than expected.

In various embodiments, one or more elements of radar chart 300 may be presented as color-coded visual elements in order to be more rapidly distinguishable. For example, if a summary score is higher than expected (e.g., suggesting that one or more patient status criteria of a relevant criteria category have a higher-than-normal value), a point corresponding to the summary score may be displayed in a red (or different) color. Additionally, a portion of a ring in which the point is located may be colored in a similar color or shade of the color (e.g., a different tone of red). If a summary score is within an expected or normal range (e.g., suggesting that the patient status criteria of a relevant criteria category have normal values), a point corresponding to the summary score may be displayed in a white (or different) color, and a corresponding ring or rings may be colored in a similar color or shade of the color (e.g., a different tone of white). As a result, a caregiver may be able to quickly scan the radar chart 300 to determine whether any patient status criteria in any of the represented criteria categories is within a normal or expected range. In contrast, in an alternative UI where radar chart 300 is not displayed, such as in conventional patient monitor displays, the individual patient status criteria may be displayed as values, for example, in tiles in a grid-type format. To determine whether any of the individual patient status criteria are outside a desired range, the caregiver may have to read the values, scrolling or paging through the grid-type format until a desired set of criteria has been checked. Additionally, in such a display, the individual patient status criteria may not be grouped, or may be grouped in ways that are not helpful in quickly assessing patient status (e.g., alphabetically, historically, etc.). As a result, the caregiver may spend more time reviewing the patient status criteria in the alternative UI than when reviewing the patient status criteria in radar chart 300. Additionally, processing and memory resources consumed during retrieving and displaying the physiological patient data may be higher when reviewing the patient status criteria in the alternative UI than when reviewing the patient status criteria in radar chart 300, since other patient data that is not relevant to the caregiver may be displayed.

An advantage of the radar chart 300 is that in various embodiments, the different criteria categories and visual elements may be configured by the user. For example, a caregiver may establish which specific categories of patient status criteria are displayed in radar chart 300, and/or which specific patient status criteria are included in each criteria category. The caregiver may specify the categories and patient status criteria based on a single patient, or a group of patients. For example, the caregiver may be monitoring a patient with a specific health concern. The caregiver may configure a first patient status visualization to have a radar chart with select patient status criteria and criteria categories that relate to the specific health concern, and may exclude patient status criteria and criteria categories that do not relate to the specific health concern. The caregiver may monitor the first patient status visualization periodically to ensure that the selected patient status criteria are maintained within desired value ranges. The caregiver may configure a second patient status visualization to have a radar chart format with different patient status criteria and criteria categories that relate to a group of patients in an ICU. For example, the caregiver may be monitoring the group of patients to determine which patient of the group of patients may be downgraded to a different care unit. The caregiver may include patient status criteria in the second patient status visualization that may be used to evaluate a patient downgrade status, which may be different from the patient status criteria included in the first patient status visualization. The caregiver may configure the second patient status visualization such that the caregiver can compare different configurations of points (e.g., points 310, 312, 314, and 316) of the radar charts of each patient of the group of patients. For example, the radar charts may be displayed adjacent to each other in the second patient status visualization, such that the caregiver may quickly distinguish patients within a normal range of patient status criteria values from patients outside the normal range of patient status criteria values.

The physiological patient data displayed in radar chart 300 may include real-time physiological patient data acquired, for example, via sensors of a patient monitor such as patient monitor 110 of FIG. 1 and/or patient monitoring devices 224 of FIG. 2. Additionally or alternatively, the physiological patient data displayed in radar chart 300 may include historical physiological patient data retrieved from a hospital information system such as computer systems and databases 210 and/or EMR database 222. In other words, radar chart 300 may be configured to include certain patient status criteria as described above, and the values or measurements associated with each patient status criteria may be displayed from the patient monitor or from the hospital information system depending on a selection of the user.

The historical values or measurements associated with each patient status criteria may be retrieved at a time of configuration, and stored in a memory of the patient status visualization system (e.g., memory 204). At a subsequent time, or at various times after configuration, the caregiver view either the real-time physiological patient data or the historical physiological patient data. The caregiver may adjust a control element of the patient status visualization (or UI within which the patient status visualization is displayed) to a first setting or position to view the real-time physiological patient data, and adjust the control element to a second setting or position to view the historical physiological patient data. Because the historical physiological patient data is retrieved at the time of configuration and stored in the memory, the historical physiological patient data may be displayed “on-demand”, meaning, without retrieving the historical physiological patient data from the hospital information system at the subsequent time.

Further, historical physiological patient data corresponding to a plurality of times in the past may be used to create a respective plurality of radar charts that all share the same configuration. As a result, the caregiver may select to view the real-time physiological patient data, or to view one or more of the respective plurality of radar charts. In some embodiments, the caregiver may adjust a control such as a slider to view a progression of physiological patient from an earliest time of the plurality of times, to a current time (e.g., when the real-time physiological patient data is displayed). In this way, the caregiver may more easily determine, for example, whether current physiological patient data is within an expected range for a specific patient.

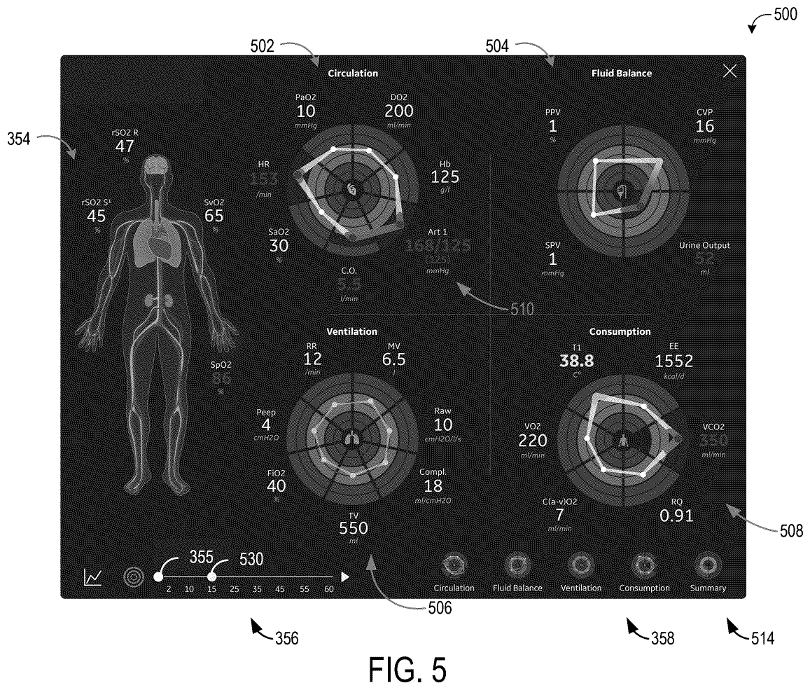

Further, visual elements of radar chart 300 may be controls (e.g., buttons) that may be selected by a user via a user input device (e.g., a mouse, a touchscreen, etc.). When the user selects an element of radar chart 300, additional information relevant to the patient status may be displayed, for example, in an additional display panel of the UI, or a pop-up display panel, as shown in the examples of FIGS. 5-8 described below. The additional information may include more detailed information, such as individual values of patient status criteria included in a relevant criteria category. The additional information may also include other, alternative representations of the patient status data, such as other graphical depictions.

For example, FIG. 3B shows an alternative patient status visualization 350, including a radar chart 352, which may be a non-limiting example of radar chart 300, and an body visualization 354. Body visualization 354 shows a generic figure 370 of a body. Generic figure 370 may include depictions of one or more body systems of the body, which may correspond to patient status criteria categories of radar chart 352. In the depicted embodiment, generic figure 370 includes representations of a respiratory system 374, and a circulatory system 376. Respiratory system 374 and circulatory system 376 may be distinguished by different colors.

Additionally, various patient status criteria values 372 may be displayed at various locations around generic figure 370. The patient status criteria values 372 may correspond to a selected criteria category of radar chart 352. For example, a user of patient status visualization display 350 may select a first patient status criteria category associated with a respiratory system (e.g., third criteria category 306 of FIG. 3A), and patient status criteria values of the respiratory system may be displayed on or around generic figure 370. The user may then select a second patient status criteria category associated with a circulatory system (e.g., first criteria category 302), and patient status criteria values of the circulatory system may be displayed on or around generic figure 370. In other embodiments, different user interactions may cause different types of patient status criteria values or related patient information to be displayed in conjunction with generic figure 370.

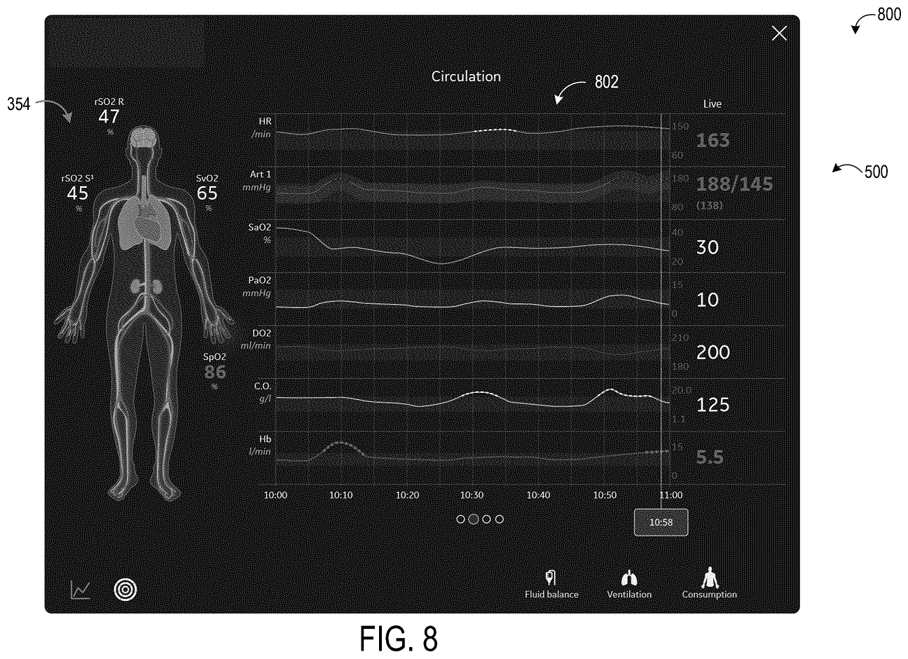

Patient status visualization 350 includes one or more toggle icons that may allow the user to switch between different visualizations of the patient status. For example, the user may select a first toggle icon 362 that may cause body visualization 354 to disappear, and radar chart 352 to be visualized (e.g., over a larger area), or the user may select a second toggle icon 364 that may cause body visualization 354 to disappear, and an alternative graphical display of the patient status data to be visualized, as shown in FIG. 8. When body visualization 354 disappears, a toggle icon for viewing body visualization 354 may be displayed in place of a toggle icon representing a currently-viewed visualization.

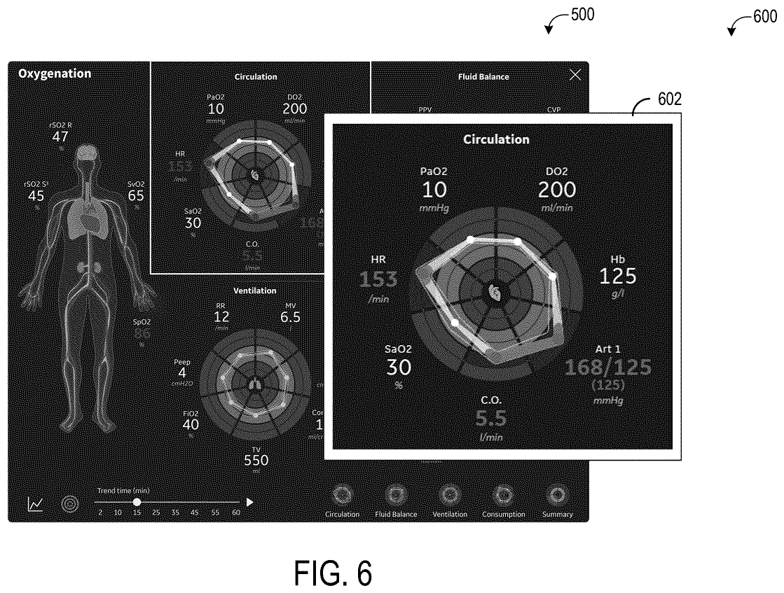

Patient status visualization 350 may include additional selectable elements or icons that may cause other visualizations to be displayed. For example, a first set of controls 358 may allow the user to view individual radar charts corresponding to criteria categories of radar chart 352 (e.g., to drill down and see individual patient status criteria values), as shown in FIG. 6. A group display control 360 may allow the user to view the radar charts corresponding to the criteria categories of radar chart 352 in a group view, as shown in FIG. 5.

Patient status visualization 350 further includes an interactive timeline slider 356, which may allow the user to view historical physiological patient data, as described above. That is, historical measurements of the patient status criteria included in radar chart 352 may be retrieved at a time of configuring radar chart 352 and used to generate a plurality of historical radar charts corresponding to different times. For example, a first historical radar chart may be generated with the same configuration as radar chart 352 but with historical patient status criteria values acquired at a first time; a second historical radar chart may be generated with the same configuration as radar chart 352 but with historical patient status criteria values acquired at a second, later time; a third historical radar chart may be generated with the same configuration as radar chart 352 but with historical patient status criteria values acquired at a third, later time; and so on. The historical radar charts may be associated with different points on timeline slider 356, such that the first historical radar chart corresponding to the first time may be visualized by the user by positioning a slider element 355 of timeline slider 356 at a first point along timeline slider 356; the second historical radar chart corresponding to the second time may be visualized by the user by positioning slider element 355 at a second point along timeline slider 356 (e.g., further to the right, in the depicted embodiment); the third historical radar chart corresponding to the third time may be visualized by the user by positioning slider element 355 at a third point along timeline slider 356; and so on. Radar chart 352 may be associated with an initial point of timeline slider 356 (as depicted in FIG. 3B), such that the user may select the initial point to view radar chart 352, which may show real-time physiological patient data of the patient being collected at a current time. In this way, the user may slide slider element 355 from right to left to view a chronological progression of the physiological patient data up to the current time, and the user may slide slider element 355 from left to right to view a reverse chronological progression of the physiological patient data from the current time. By viewing the physiological patient data over time in this way, the user may more quickly and efficiently detect deviations in the physiological patient data over time than in an alternative UI not including timelines slider 356.

Further, in some embodiments, timeline slider 356 may be further configured to allow the user to set an amount of time over which the radar chart 352 summarizes the physiological patient data. For example, timeline slider 356 may include a set of markings 380 indicating a number of days. The user may position slider element 355 at the initial point to view real-time physiological patient data of the patient at the current time. The user may position slider element 355 at a point 381 on timeline slider 356 to view average physiological patient data collected over the previous 15 days; or the user may position slider element 355 at a point 382 on timeline slider 356 to view average physiological patient data collected over the previous 45 days. To generate the average physiological patient data, the real-time physiological patient data may be averaged with the historical physiological patient data stored in the memory of the patient status visualization system.

Referring now to FIG. 4A, a method 400 is shown for generating patient status visualizations for a selected plurality of patients, according to an embodiment of the disclosure. Method 400 may be carried out by a patient status visualization system, such as the patient status visualization system 220 of FIG. 2. Method 400 may be performed according to instructions stored in a memory (e.g., memory 204) of the patient status visualization system, which may be executed by a processor (e.g., processor(s) 206) of the patient status visualization system. In various embodiments, the patient status visualization system may be integrated into or launched by a software or web-based application used by caregivers for viewing real-time physiological patient data. For example, a caregiver may view the real-time physiological patient data in a first format in a first display panel of the application, and the patient status visualization system may be launched in response to the caregiver selecting a control element of the application (e.g., a button, etc.) to request a patient status visualization. The requested patient status visualization may be configured by the caregiver to convert the real-time physiological patient data into a second, different format for displaying the patient status visualization, and the patient status visualization may be displayed in a second display panel of the application. In other embodiments, the patient status visualization system may be launched by the caregiver, and the patient status visualization may be displayed in a display panel of the patient status visualization system.

Method 400 begins at 402, where method 400 includes receiving a request to generate one or more patient status visualizations (also referred to herein as a status visualization request). The patient status visualizations may be generated for a single patient, or a plurality of patients of a care unit of a healthcare organization or network (e.g., a hospital), such as an ICU. In various embodiments, the status visualization request may be initiated automatically by the patient status visualization system, such that patient status visualizations are generated for eligible patients and updated on a regular basis (e.g., each minute). Alternatively, the status visualization request may be initiated by a caregiver or other user.

To initiate the status visualization request, the user may open a patient status visualization system application running on a computer or network terminal of the ICU or on the patient monitor and select one or more suitable controls of a UI of the patient status visualization system (e.g., a clinician may select a user interface button displayed on a display device to request patient status visualizations to be displayed). In some embodiments, the user may initiate a status visualization request as part of a routine procedure, (e.g., a daily procedure) in accordance with one or more clinical guidelines of the healthcare organization, as part of routine patient management practices. In other embodiments, the user may initiate a status visualization request in response to a desire to assess the status of a specific patient.

At 404, method 400 includes receiving a selection of patients to analyze to generate the patient status visualizations. In some embodiments, the patient status visualization system may be hosted on a server (e.g., of the healthcare organization) and accessible to a plurality of care units via a network, and the user may enter in a name of a patient of a care unit, or select the care unit from a list of care units in order to indicate the selection of patients. In other embodiments, a relevant care unit may automatically be selected based on a pre-defined configuration file created during a setup stage of the patient status visualization system.

At 406, method 400 includes generating a patient status visualization for each patient of the selection of patients. Generating the patient status visualization for each patient may include retrieving clinical information of the patient from various sources and processing the information, as described in greater detail below in reference to FIG. 4B.

At 408, method 400 includes displaying the patient status visualizations of the selected patients on the display screen of the patient status visualization system. The patient status visualizations may be presented on the screen in a UI (e.g., UI 214) of the patient status visualization system, for example, as described in reference to FIGS. 3A and 3B.

At 410, method 400 includes determining whether the user has selected a visualization element of the patient status visualization. The user (who may be a caregiver) may select the visualization element via a mouse or touch hover, or a mouse click or touch input, to view additional information associated with the patient status visualization. The additional information may include more detailed patient status data than is displayed at a top level of the patient status visualization, such as values or measurements of individual patient status criteria, as described above. If the user has selected a patient status visualization or element, method 400 proceeds to 412 to display the additional information based on the selected visualization element.

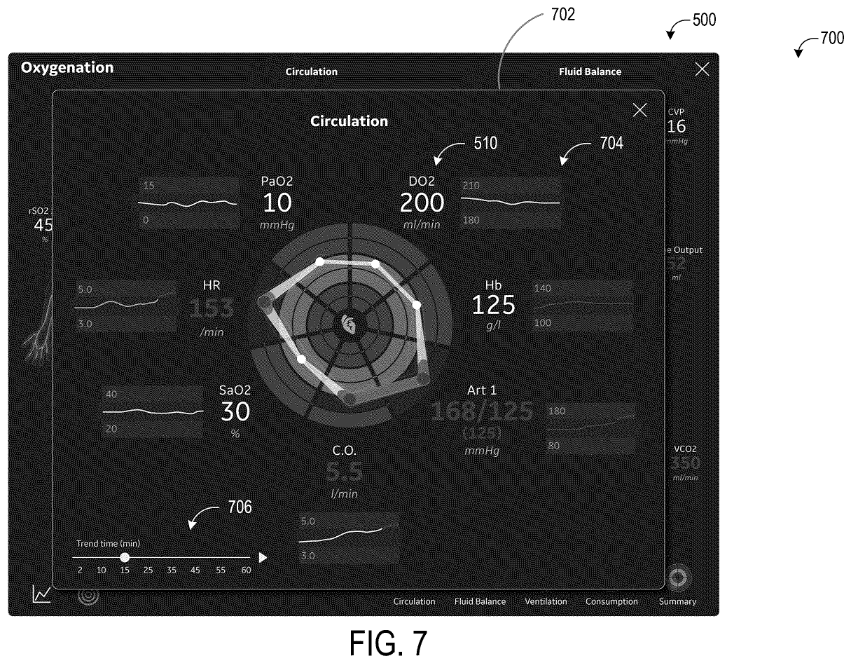

Displaying the additional information may include displaying an additional display panel in the UI with the additional information. The additional display panel may be pop-up display, such as the pop-up display panels shown in FIGS. 6 and 7. In some examples, displaying the additional information may include displaying an additional or alternative visualization element to a currently-displayed visualization element. For example, the patient status visualization may include a first visualization element, such as a radar chart (e.g., radar chart 300 of FIG. 3A). The user may select a control element of the patient status visualization (e.g., toggle icons 362 and 364) to additionally or alternatively display a second visualization element, such as a body visualization of patient status criteria data (e.g., body visualization 354 of FIG. 3B), or a different visualization.

Displaying the additional information may also include displaying more detailed patient status information associated with one or more criteria categories of the patient status visualization. For example, the patient status visualization may include the radar chart, which may comprise four criteria categories, as described in relation to FIG. 3A. The user may select a control element (e.g., group display control 360 of FIG. 3B) to view more detailed radar charts for each criteria category of the four criteria categories, as shown in FIG. 5.

Alternatively, if at 410 the user has not selected a patient status visualization or element, method 400 proceeds to 414. At 414, method 400 includes determining whether the user has adjusted a timeline slider (e.g., timeline slider 356) of the patient status visualization, to view historical physiological patient data rather than or along with the real-time physiological patient data. If at 414 it is determined that the user has adjusted the timeline slider, method 400 proceeds to 416.

At 416, method 400 includes integrating the historical physiological patient data into the display of the patient status visualization. A position of a slider element (e.g., slider element 355) of the timeline slider is received, which indicates a desired point in time in the past. For example, the desired point in time in the past may be a week prior to a current time, or two weeks, or a month, or two months, or a different point in time. In one embodiment, historical physiological patient data from the point in time is retrieved from the memory of the patient status visualization system, and the historical physiological patient data from the point in time is displayed within a historical radar chart having a same configuration as the radar chart showing the real-time physiological patient data. That is, the historical radar chart may have the same criteria categories and same patient status criteria within the criteria categories as the radar chart showing the real-time physiological patient data. The historical radar chart may be displayed in a same location of a same display panel as the radar chart showing the real-time physiological patient data, such that the radar chart showing the real-time physiological patient data may be replaced by the historical radar chart. Thus, when the user adjusts the position of the slider element, the conical sections and concentric rings of the radar chart showing the real-time physiological patient data may appear to remain on the screen, while the locations of the points and lines forming a first network shape of the real-time radar chart may appear to be replaced by new points and lines forming a second network shape of the historical radar chart. In this way, a difference between the first network shape and the second network shape may be accentuated. If the difference is small, it may be inferred by a caregiver that a status of the patient may be similar to a prior status of the patient at the point in time. If the difference is larger, it may be inferred that the status of the patient may have changed since the point in time. In some cases, the caregiver may select the historical radar chart to view individual patient status criteria values associated with one or more criteria categories of the historical radar chart, whereby method 400 may proceed back to 410.

In another embodiment, when the slider element position is received, historical physiological patient data collected between the current time and the point in time may be retrieved from the memory of the patient status visualization system, and the historical physiological patient data may be aggregated with the real-time physiological patient data and averaged, and average values for each patient status criterion may be displayed within a combined radar chart having a same configuration as the radar chart showing the real-time physiological patient data. For example, a caregiver may wish to view the real-time physiological patient data, and then compare the real-time physiological patient data with average historical values for the patient. In still other embodiments, the historical physiological patient data may be processed and displayed within the patient status visualization in a different manner.

If at 414 it is determined that the user has not adjusted the timeline slider, method 400 proceeds to 418, where method 400 includes continuing to display the patient status visualization. Continuing to display the patient status visualization may include updating the patient status visualization as real-time physiological patient data is generated, and/or as real-time physiological patient data is stored as historical physiological patient data. Method 400 then ends.

Referring now to FIG. 5, an example of a patient status visualization 500 of a patient is shown, which may be a secondary patient status visualization generated from patent status visualization 350 of FIG. 3B that shows additional information related to radar chart 352. Patient status visualization 500 may be displayed in response to a user selecting group display control 360 of FIG. 3B. When the user selects group display control 360, radar chart 352 may be replaced with four patient status criteria radar charts, one for each of the criteria categories of radar chart 352 (e.g., radar chart 300 of FIG. 3A). The four patient status criteria radar charts include a first patient status criteria radar chart 502, corresponding to patient status criteria included in first criteria category 302 of radar chart 300 (e.g., circulation); a second patient status criteria radar chart 504, corresponding to patient status criteria included in second criteria category 304 of radar chart 300 (e.g., fluid balance); a third patient status criteria radar chart 506, corresponding to patient status criteria included in third criteria category 306 of radar chart 300 (e.g., ventilation); and a fourth patient status criteria radar chart 508, corresponding to patient status criteria included in fourth criteria category 308 of radar chart 300 (e.g., consumption). Additionally when the user selects group display control 360, group display control 360 may be replaced by a summary view control 514, which when selected by the user may replace the display of the four patient status criteria radar charts with radar chart 352 (e.g., a high-level summary radar chart).

Each of patient status criteria radar charts 502, 504, 506, and 508 may be configured similarly to radar chart 300, where each patient status criteria radar chart may include a plurality of points corresponding to individual patient status criteria values associated with a corresponding criteria category. For example, in the depicted embodiment, first criteria category 302 includes seven patient status criterion that may be used to assess the status of the patient with respect to the patient's circulatory system: partial pressure of oxygen (PaO2), delivered oxygen (DO2), hemoglobin (Hb), arterial pressure (Art 1), cardiac output (C.O.), arterial oxygen saturation (SaO2), and heart rate (HR). For each of the seven patient status criterion, a measurement value 510 may be indicated, which may be used to determine a placement of a point corresponding to the measurement value 510 within a corresponding conical section of patient status criteria radar chart 502, as described above in reference to FIG. 3A. The points may be connected by lines to form a network shape, and the points, lines, and/or concentric rings of patient status criteria radar chart 502 may be colored, shaded, or highlighted based on a corresponding measurement value 510.

By presenting the patient status criteria measurement values as points in radar chart 502, the user may more quickly and efficiently determine whether one or more patient status criteria values is outside a range of expected or desired values, whereby medical attention may be warranted, than if the patient status criteria measurement values were displayed in an alternative manner, such as in a list or a grid view. That is, the radar chart may leverage a natural human ability to quickly recognize shapes, and deviations from shapes, at a pre-cognitive stage, meaning without entailing mental concentration or computation. Rather, any points deviating from the network shape “pop out”, forming angles in the shape of the network that advantageously draw the user's attention to a location of a corresponding measurement value 510, which may be highlighted, for example in a color such as red. As a result, an amount of time taken by the user to assess the status of the patient based on the measurement values 510 may be less than a second amount of time taken to review the measurement values 510 in the alternative manner.

Further, by displaying the patient status criteria radar charts 502, 504, 506, and 508 together in a single display panel, a larger overall number of measurement values 510 may be assessed. For example, by scanning the patient status criteria radar charts 502, 504, 506, and 508, the user may rapidly determine that measurement values for the HR, Art. 1, and C.O. criteria of the circulation category, a measurement value for VCO2 of the oxygen consumption category, and a urine output of the fluid balance category are outside of an expected or desired range of values; and that measurement values for criteria of the ventilation category are within an expected or desired range of values.

As previously noted, the measurement values 510 included in patient status criteria radar charts 502, 504, 506, and 508 may correspond to real-time physiological patient data or historical physiological patient data. As with radar chart 300, the user may view measurement values 510 acquired at different times, by adjusting a position of slider element 355 within timeline slider 356. For example, slider element 355 may be positioned at the initial point of timeline slider 356 to view the real-time physiological patient data visualized in patient status criteria radar charts 502, 504, 506, and 508, or slider element 355 may be positioned at a second point 530 of timeline slider 356 to view the historical physiological patient data from a specific time, or average physiological patient data over a specific time period visualized in patient status criteria radar charts 502, 504, 506, and 508.

Each of patient status criteria radar charts 502, 504, 506, and 508 may be selectable by the user. In some embodiments, in response to selecting a patient status criteria radar chart, the selected patient status criteria radar chart may be displayed in a pop-up display panel. For example, FIG. 6 shows a first pop-up example 600 including a pop-up display panel 602, where patient status criteria radar chart 502 is displayed in pop-up display panel 602. Pop-up display panel 602 may be a resizable display panel that allows the user to increase a size of patient status criteria radar chart 502. For example, as a result of a number of patient status criteria radar charts of patient status visualization 500 being high, a size of each patient status criteria radar chart of patient status visualization 500 may be small, whereby the user may increase the size of a patient status criteria radar chart of patient status visualization 500 by selecting the patient status criteria radar chart and viewing the patient status criteria radar chart in pop-up display panel 602.

Pop-up display panel 602 may also be configured and/or customized by the user to include additional or alternative display elements that may show more detailed patient status information. For example, FIG. 7 shows a second pop-up example 700 including a customized pop-up display panel 702, where patient status criteria radar chart 502 is displayed in customized pop-up display panel 702. Customized pop-up display panel 702 additionally displays, for each patient status criteria measurement value 510, a graph 704 showing a trend in the patient status criteria measurement value 510 over a specified time period, where the specified time period may be selected via a timeline slider 706 similar to timeline slider 356 of FIGS. 3B and 5. In other embodiments, a different type of graphical element or visualization may be included in pop-up display panel 702.

In other embodiments, in response to selecting a patient status criteria radar chart, an alternative visualization of information shown in the patient status criteria radar chart may be displayed within patient status visualization 500. For example, FIG. 8 shows an alternative configuration 800 of patient status visualization 500, where patient status criteria radar charts 502, 504, 506, and 508 have been replaced with a graph 802 corresponding to patient status criteria radar chart 502. Graph 802 may be displayed in patient status visualization 500 in response to the user selecting patient status criteria radar chart 502 of the circulation category, for example. Graph 802 includes trend graphs for each patient status criteria included in the circulation category, similar to the graphs 704 of FIG. 7. As in FIGS. 3A and 3B, trend graphs that include values that are outside the expected or desired range of values may be highlighted using coloring, shading, etc.

Referring now to FIG. 4B, a method 450 for generating a patient status visualization for a patient is shown. Method 450 may be carried out by a patient status visualization module of a patient status visualization system, such as patient status visualization module 240 of patient status visualization system 220, according to instructions stored in memory (e.g., the memory 204), which may be executed by a processor (e.g., processor(s) 206) of the patient status visualization system. Method 450 may be performed as part of method 400 described above in reference to FIG. 4A.

Method 450 begins at 452, where method 400 includes receiving identifying information of a patient for which a patient status visualization is to be made. In some embodiments, the patient status visualization system may iteratively generate and update patient status visualizations for each patient in a given hospital unit, such as an ICU. When the patient status visualization system generates a patient status visualization for a given patient, the patient status visualization system may input an identifier of that patient into the patient status visualization module, to receive, as an output of the patient status visualization module, a patient status visualization for the relevant patient. Alternatively, a caregiver may submit identifying information of one or more patients in a request for a patient status visualization.

At 454, method 450 includes receiving a set of patient status criteria categories relevant to a status of the patient. In some embodiments, the set of patient status criteria categories may be specified by the user, via an input device. For example, the user may select the set of patient status criteria categories from a menu of the patient status visualization system. In other embodiments, the set of patient status criteria categories may be configured in a pre-defined manner by a health care organization (e.g., a hospital) or by a manager of a particular care unit, or by a different entity or individual. For example, the set of patient status criteria categories may be configured based on a type of care unit in which the patient is receiving care. As there may be different types of care units within a healthcare organization, each care unit may include different patient status criteria that may affect what patient data is analyzed to determine the patient status. For example, a first set of physiological patient data may be obtained and analyzed by a first user to determine whether an adult patient may be released from a first care unit, and a second set of physiological patient data may be obtained and analyzed by a second user to determine whether a baby may be released from a neonatal ICU. One advantage to configuring the set of patient status criteria categories in the pre-defined manner is that training of caregivers may be easier and/or more efficient. For example, the caregivers may be trained to expect a consistently organized set of visualizations, where criteria categories and patient status criteria values are always displayed in a same part of a display panel of the patient status visualization.

At 456, method 450 includes receiving a set of patient status criteria to be included in each criteria category. In some embodiments, the set of patient status criteria may be specified by the user, for example, via a menu of the patient status visualization system. In other embodiments, the set of patient status criteria may be configured in a pre-defined manner by the health care organization, care unit, or a different entity or individual. In some examples, the set of patient status criteria may be determined based on a set of clinical guidelines. Further, in other embodiments, the set of patient status criteria may be configured automatically by an artificial intelligence (AI) or machine learning (ML) model of the patient status visualization system, based on input data provided by the user. For example, the set of patient status criteria may be determined based on an output of a rules-based system developed based on statistical or historical patient data.

At 458, method 450 includes retrieving historical physiological patient data of the patient from one or more clinical information systems. Once the patient status criteria have been determined, stored measurement values of each criterion over a period of time may be retrieved. The period of time may be pre-determined, based on clinical guidelines, for example, or may be configured by the user. In one example, the period of time may be one month, or 60 days. A number of stored measurement values for each criterion may vary; for some criteria, historical physiological patient data may be available for more frequent time intervals than for other criteria.

Further, the patient status visualization module may analyze the plurality of records to determine one or more trends in vital signs or clinical markers, where the one or more trends may be used in a graphical display element such as graph 802 of FIG. 8. In some examples, the patient status visualization system may receive a data feed from the EMR that includes patient parameters (e.g., vital signs, ventilation status, etc.) dictated by the patient status criteria for the patient. By obtaining the patient parameters from a single source on a semi-continuous feed (e.g., updated every 10, 15, or 30 seconds), the efficiency of the patient status visualization system may be improved by reducing processing demands and network traffic associated with obtaining the patient parameters from multiple sources and/or requesting the patient parameters each time a patient status visualization is made. In some examples, the patient status visualization system may map specific feeds for each data source from the EMR using an API, which may be performed for each criterion.

At 460, method 450 includes receiving real-time physiological patient data corresponding to the set of patient status criteria from one or more patient monitors, and at 462, method 450 includes converting the real-time physiological patient data in each criteria category of the patient status visualization into a criteria category score (e.g., the first circulatory score, the second fluid balance score, the third ventilation score, and the fourth oxygen consumption score of FIG. 3A). The criteria category scores may be used to position points on a radar chart of the patient status visualization, as described in reference to FIG. 3A.

At 464, method 450 includes generating one or more visualization elements of the patient status visualization, based on the received/retrieved physiological patient data and/or the criteria category scores. The visualization elements may include a summary radar chart that depicts a summary of the criteria category scores, such as radar chart 300. The one or more visualization elements may also include a body visualization (e.g., body visualization 354), a graph visualization (e.g., graph 802), and/or a different visualization. For each score calculated, ranges may be established that are configurable or follow physiological/measurement maximum/minimum values. Upper and lower target zones (thresholds) may be configurable based on patient needs, and can be tied to care targets, set alarm limits, etc. A position of each score within a respective range may then be used to determine a placement of a visual element indicating the score in a visualization, as described in greater detail below. In some cases, scores may be assigned relative weights.

At 466, method 450 includes outputting the patient status visualization for display on a display device, and method 450 ends.

As an example of how the patient status visualization system might work, a caregiver at an ICU of a hospital may wish to review the status of a first patient of the ICU on a networked computer of the ICU. The caregiver may log into a UI of a hospital network on the networked computer, and the caregiver may select a first patient to view a first set of physiological patient data (e.g., vital signs, acuity scores, etc.) collected in real time from the first patient from sensors of a first patient monitor arranged on the first patient. Information of the first patient may be displayed in a display panel of the UI.

The caregiver may launch the patient status visualization system via a button of the a display panel. The patient status visualization system may receive the first set of physiological patient data of the first patient from the first patient monitor. Based on the location of the patient (e.g., the ICU), the patient status visualization system may retrieve a configuration template for the ICU from a memory of the patient status visualization system, and may configure a first patient status visualization of the first set of physiological patient data based on the configuration template. The configuration template may be previously created by the caregiver, or a different person. The configuration template may define a number of patient status criteria to display in the first patient status visualization, which may be divided into a plurality of categories. The first patient status visualization may include a first summary radar chart (e.g., radar chart 300) that summarizes the status of the first patient, based on the received first set of physiological patient data.

The caregiver may view a first network shape of lines and points of the first summary radar chart that summarize the patient status. The first network shape may be a symmetrical shape, where the points of the first summary radar chart appear within expected ranges for the first patient, where the expected ranges are established by a set of clinical guidelines of the ICU. As a result of the points of the first summary radar chart appearing within expected ranges, the caregiver may infer that the first patient is stable.

The caregiver may then select a second patient to view a second set of physiological patient data collected in real time from a second patient monitor arranged on the second patient. Information of the second patient may be displayed in the display panel of the UI. The caregiver may launch the patient status visualization system via the button of the display panel. The patient status visualization system may receive the physiological patient data of the second patient from the patient monitor, and may configure a second patient status visualization of the second set of physiological patient data based on the configuration template. The second patient status visualization may include a second summary radar chart that summarizes the status of the second patient, based on the received second set of physiological patient data. The patient status visualization system may display the second patient status visualization in the UI. Additionally, and concurrently, the patient status visualization system may retrieve historical physiological patient data of the second patient corresponding to the second set of physiological patient data from one or more systems and/or databases of the hospital. The historical physiological patient data may be retrieved in the background while the caregiver is viewing the real-time physiological patient data, and stored in a memory of the patient status visualization system.

The caregiver may view a second network shape of lines and points of the second summary radar chart that summarize the patient status of the second patient. The second network shape may be an asymmetrical shape, where a point of the second summary radar chart corresponding to a circulatory system of the second patient may appear outside an expected range for the second patient. For example, the point may appear closer to an outer perimeter (e.g., outer perimeter 348) than a center point (e.g., center point 340) of the second radar chart. As a result of the point of the second summary radar chart appearing outside of the expected range, the caregiver may wish to view patient data of the second set of physiological patient data associated with the circulatory system of the second patient. The caregiver may select a quadrant of the second summary radar chart associated with the circulatory system using an input device of the networked computer.

When the caregiver selects the quadrant associated with the circulatory system, the second summary radar chart may be replaced in the UI by a circulation radar chart, such as radar chart 502 of FIGS. 5 and 6. The caregiver may view a third network shape of lines and points of the circulation radar chart. The third network shape may be an asymmetrical shape, where a point of the second summary radar chart corresponding to a heart rate (HR) of the second patient may appear outside an expected HR range for the second patient. A measured value of the individual patient status criteria may be displayed near the point. The caregiver may confirm that the measured value is outside the expected HR range.

The caregiver may then wish to view historical HR data of the second patient, to determine a baseline HR of the second patient. The caregiver may adjust a slider element of a timeline slider of the second patient status visualization (e.g., slider element 355 of timeline slider 356) from a first position at a first end of the timeline slider to a second position of the timeline slider corresponding to 30 days prior to a current date. The caregiver may select, for example, from a right-click dialog box of the UI, to view average HR data of the second patient over the selected period of time (e.g., 30 days). The patient status visualization system may retrieve historical physiological patient data of the second patient from the memory of the patient status visualization system. The patient status visualization system may calculate an average of the historical HR of the second patient over the selected period of time, including the current HR data received from the second patient monitor. The average HR of the patient may be displayed in the display panel of the UI.

The caregiver may see that the average HR of the patient is higher than expected. As a result, the caregiver may wish to view a trend in the HR over the period of time, to determine whether the HR has been trending up or whether the HR has been maintained at the higher-than-expected value. The caregiver may select, for example, from the right-click dialog box of the UI, to view individual HR data values of the second patient over the selected period of time (e.g., 30 days). The caregiver may then slide the slider element from the second position back to the first position of the timeline slider. As the caregiver slides the slider element, the circulation radar chart may be adjusted in the display panel of the UI based on a current position of the slider element. In other words, as the slider element is moved past a position on the timeline slider corresponding to a date with the period of time, the third network shape of the circulation radar chart may be modified to match the HR data of the second patient at the data. In this way, the caregiver may view a progression of the HR data of the second patient over time from the second position of the timeline slider to the first position of the timeline slider, in an animated fashion. As the caregiver slides the slider element, the caregiver may see that the HR of the second patient has increased over the period of time.

The caregiver may then select the circulation radar chart, and via the right-click menu, may select to display a trend graph of the patient status criteria displayed in the circulation radar chart. In response, the patient status visualization system may replace the circulation radar chart in the display panel with a trend graph, such as graph 802 of FIG. 8. The caregiver may view the trend graph, and may see a rate at which the HR of the second patient is increasing. Based on the trend graph, the caregiver may set an alert to notify members of the ICU if the measured value of the HR of the second patient increases above a threshold HR.

In this way, the patient status visualization system and UI elements provided herein may allow the caregiver to more rapidly and efficiently determine whether measured values of physiological patient data of a patient are within acceptable ranges, than in an alternative UI in which the patient status visualization is not displayed. As a result, an accuracy and timeliness of patient monitoring may be increased, generating faster response times to changes in patient status and a more efficient process for initiating downgrades or transfers out of high-demand, high-resource hospital units, which may reduce costs for the patient and for a healthcare system. Additionally, historical physiological patient data may be displayed on demand in an intuitive, efficient manner, where the historical physiological patient data is retrieved from various systems and databases of the healthcare system at a first time during a configuration of the patient status visualization, such that the historical physiological patient data may be visualized in real time, on demand, during consultation of the patient data at a later time via the patient status visualization. By establishing a system that automatically retrieves data from various EMRs in real time or near real time, aggregates that information regardless of data format, and converts the retrieved data into a standardized format that is saved locally for viewing on demand, the approach of the disclosure allows caregivers to make informed decisions about patients, such as when to provide treatment, when to transfer patients, and where to transfer the patients, thereby improving patient care.

In contrast, in prior systems when the caregiver attempted to assess the status of a patient, errors could be made due to delays in obtaining desired parameters for evaluating the patient status. For example, a patient's condition may have improved to the point where the patient would be ready for a downgrade to a medical-surgical unit rather than an ICU, but limited caregiver resources may result in the patient being kept in the ICU for 12, 24, or more hours than necessary, thereby utilizing ICU resources unnecessarily. The described patient status visualization system solves this problem by providing an efficient tool for viewing both current and historical patient data in a holistic manner. In this way, the patient status visualization system provides an improvement to the capability of the healthcare system as a whole. The disclosure provides a specific way of improving the capability of the healthcare system, by providing one or more patient status visualizations that display dynamically updated physiological patient data. The disclosure further provides a specific improvement to the way computers operate by aggregating EMR data for one or more patients in one local memory, and updating the patient status visualizations in real-time, which may obviate the need for users to have to navigate through multiple different data files, manually update information as availability changes, and so forth, thereby increasing the efficiency of the operation of the computer for the user.

The patient status visualization system described herein provides a specific manner of displaying a limited set of information to a user (summary patient data), rather than using conventional user interface methods to display a generic list or view of the patient data on a computer, requiring the user to step through many layers of menu options to reach the desired data, or burying the desired data within all hospital data. Thus, the user experience with the computer may be improved and made more efficient.

Furthermore, by displaying a limited set of information via the patient status visualization as described herein, operation of the computing device(s) that collect and render the data for display may be improved by reducing the processing demands of the computing device(s), thereby increasing the efficiency of the computing device(s). For example, only certain causal criteria leading to a specific patient status visualization may be displayed, which results in a limited amount of the data that is received being processed, which may improve the efficiency of the computing device(s). The certain causal criteria may be defined and customized by the caregiver. Further, in some examples, the data is processed in real time and updates to the patient status visualization are made continuously as data is received, and therefore undue processing lags that may occur if updates were made at predefined discrete time points may be reduced, which may improve the efficiency of the computing device(s).

Thus, via the disclosed patient status visualization system, data relevant to a patient status may be displayed in a manner that is easy to visually parse and act on in a reduced amount of time. The patient information may be displayed via small graphical elements with minimal text, which may allow a large number of patient status visualizations to be included on the same screen. A user may then select a graphical element of interest to view more detailed information. The patient information may include historical data retrieved from different databases that would otherwise be accessed via individual interfaces. Thus, by using the patient status visualization system to aggregate the patient information, an amount of time taken to review relevant patient information for diagnosis and treatment decisions may be reduced. The patient status visualization system disclosed herein may also aggregate patient data to a single place, into a single application, which helps reduce wasted time spent searching for known but scattered data, and unknown and missing data. This reduces cognitive overloads and aids clinical thinking, because the patient data is reconstructed into a clinically helpful structure. Additionally, if network lags or outages result in historical patient data stored in an EMR database being unavailable, or if the EMR is in an unlaunched state, the locally stored historical patient data may still be displayed within patient status visualization. A limited amount of the historical patient data (e.g., a summary) may be displayed in a first visualization, and more detailed historical patient data may be directly viewed in real time by selecting portions of the first visualization. The technical effect of automatically generating patient status visualizations that summarize both real-time and historical physiological patient data in an integrated display, is that a consumption of processing and memory resources of a clinical information system may be reduced, by reducing a manual retrieval of data from various sources, and an amount of time taken by caregivers to assess a status of a patient may be reduced, leading to more timely interventions and improved outcomes.

The disclosure also provides support for a method for a patient status visualization system, the method comprising: receiving physiological patient data generated by one or more sensors arranged on a patient, in real time, retrieving historical physiological patient data corresponding to the physiological patient data from one or more systems and/or databases communicatively coupled to the patient status visualization system, and storing the retrieved historical physiological patient data in a memory of the patient status visualization system, generating a patient status visualization of a status of the patient, based on the real-time physiological patient data, displaying the patient status visualization on a display device, and in response to a user adjusting a control element of the patient status visualization, retrieving one or more elements of the historical physiological patient data from the memory and displaying the one or more elements in the patient status visualization in real time as the user adjusts the control element. In a first example of the method, the control element may be adjusted by the user to a position within a range of positions, and the one or more elements of the historical physiological patient data retrieved from the memory correspond to a point in time indicated by the position. In a second example of the method, optionally including the first example, the control element is a slider. In a third example of the method, optionally including one or both of the first and second examples, a chronological progression of the historical physiological patient data may be displayed by adjusting the position of the control element through the range of positions in a first direction, and a reverse chronological progression of the historical physiological patient data may be displayed by adjusting the position of the control element through the range of positions in a second, opposite direction. In a fourth example of the method, optionally including one or more or each of the first through third examples, the chronological progression of the historical physiological patient data may be displayed by adjusting the position of the control element through the range of positions when the one or more systems and/or databases communicatively coupled to the patient status visualization system are inaccessible or in an unlaunched state. In a fifth example of the method, optionally including one or more or each of the first through fourth examples, the method further comprises: in response to the user adjusting the control element to a position of the range of positions: retrieving one or more elements of the historical physiological patient data from the memory corresponding to a time period between a current time and a time indicated by the position, calculating an average of the retrieved one or more elements of the historical physiological patient data with the real-time physiological patient data, and displaying the average in the patient status visualization. In a sixth example of the method, optionally including one or more or each of the first through fifth examples, the method further comprises: continuously receiving the physiological patient data in real time from one or more patient monitors coupled to the one or more sensors, and updating the patient status visualization in real time as the physiological patient data is received. In a seventh example of the method, optionally including one or more or each of the first through sixth examples, the patient status visualization includes a radar chart visually depicting a status of the patient with respect to a plurality of patient status criteria categories of the physiological patient data, each criteria category including physiological patient data corresponding to a plurality of patient status criteria, the plurality of patient status criteria categories and the plurality of patient status criteria in each patient status criteria category of the plurality of patient status criteria categories configurable by the user. In a eighth example of the method, optionally including one or more or each of the first through seventh examples, the plurality of patient status criteria categories further comprises: a first category including physiological patient data corresponding to a circulatory system of the patient, a second category including physiological patient data corresponding to a respiratory system of the patient, a third category including physiological patient data corresponding to a balance of fluids of the patient, and a fourth category including physiological patient data corresponding to an oxygen consumption of the patient. In a ninth example of the method, optionally including one or more or each of the first through eighth examples, generating the patient status visualization of the status of the patient further comprises: for each criteria category of the plurality of patient status criteria categories: processing the physiological patient data of the criteria category to convert the physiological patient data into a single score for the criteria category, placing a point at a distance from a center of the radar chart in a respective portion of the radar chart corresponding to the criteria category, the distance determined based on the single score, and drawing lines between the points of the radar chart to form a network shape. In a tenth example of the method, optionally including one or more or each of the first through ninth examples, the method further comprises: color-coding one or more points and/or lines of the radar chart based on the single score. In a eleventh example of the method, optionally including one or more or each of the first through tenth examples, the method further comprises: in response to the user selecting an element of the patient status visualization, displaying more detailed information of the patient status, the more detailed information including at least one of: measurement values of physiological patient data of a criteria category, a second radar chart depicting the physiological patient data of the criteria category, a graph showing a trend in physiological patient data, and a visualization of one or more physiological patient data superimposed on a depiction of a human body. In a twelfth example of the method, optionally including one or more or each of the first through eleventh examples, the radar chart is configured to display a preview of historical physiological patient data in the patient status visualization, and the more detailed information is displayed in response to the user directly selecting the preview, while the one or more systems and/or databases communicatively coupled to clinical information system are inaccessible or in an unlaunched state.

The disclosure also provides support for a patient status visualization system, comprising: one or more processors, and a memory storing instructions executable by the one or more processors to: generate a patient status visualization of a status of a patient of a healthcare system, based on physiological patient data received in real time from one or more patient monitors coupled to the patient, retrieve historical physiological patient data corresponding to the physiological patient data from an electronic medical record (EMR) database of the healthcare system communicatively coupled to the patient status visualization system, and store the retrieved historical physiological patient data in a memory of the patient status visualization system, display the patient status visualization on a display device, and switch between displaying the real-time physiological patient data and the stored historical physiological patient data in response to a user adjusting a timeline slider element of the patient status visualization. In a first example of the system, the timeline slider element may be adjusted by the user to a position within a range of positions representing points in time, and historical physiological patient data corresponding to a point in time indicated by the position is displayed on the display device. In a second example of the system, optionally including the first example, further instructions are included in the memory that when executed, cause the one or more processors to: in response to the user adjusting the timeline slider element: retrieve elements of the stored historical physiological patient data occurring between the point in time and a current point in time, display average data values of the elements of the stored historical physiological patient data on the display device. In a third example of the system, optionally including one or both of the first and second examples, the patient status visualization includes a radar chart visually depicting the status of the patient with respect to a plurality of patient status criteria categories of the physiological patient data, each criteria category including physiological patient data corresponding to a plurality of patient status criteria, the plurality of patient status criteria categories and the plurality of patient status criteria in each patient status criteria category of the plurality of patient status criteria categories configurable by the user. In a fourth example of the system, optionally including one or more or each of the first through third examples, further instructions are included in the memory that when executed, cause the one or more processors to: for each criteria category of the plurality of patient status criteria categories: process the physiological patient data of the patient status criteria category to convert the physiological patient data into a single score for the patient status criteria category, place a point at a distance from a center of the radar chart in a respective portion of the radar chart corresponding to the patient status criteria category, the distance determined based on the single score, and draw lines connecting the points of the radar chart to form a network shape that visually summarizes the patient status. In a fifth example of the system, optionally including one or more or each of the first through fourth examples, further instructions are included in the memory that when executed, cause the one or more processors to: in response to the user selecting an element of the patient status visualization, display more detailed information of the patient status, the more detailed information including at least one of: measurement values of physiological patient data of a patient status criteria category, a second radar chart depicting the physiological patient data of the patient status criteria category, a graph showing a trend in a physiological patient data, and a visualization of one or more physiological patient data superimposed on a depiction of a human body.

The disclosure also provides support for a method, comprising: obtaining physiological patient data from one or more medical devices each monitoring a patient, grouping the physiological patient data into two or more criteria categories relating to a status of the patient, displaying, via a graphical user interface (GUI) displayed on a display screen, a first patient status summary that includes a metric for each criteria category of the two or more criteria categories derived from the physiological patient data, and responsive to user input, displaying, via the GUI, a respective second patient status summary for each criteria category of the two or more criteria categories that includes a value for each element of the physiological patient data included in the category, wherein the first patient status summary is a first radar chart that plots each metric in a different sector of the first radar chart with lines connecting each metric, and each second patient status summary is a second radar chart that plots each value in a different sector of the second radar chart with lines connecting each value.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for a patient status visualization system, the method comprising:
receiving physiological patient data generated by one or more sensors arranged on a patient, in real time;
retrieving historical physiological patient data corresponding to the physiological patient data from one or more systems and/or databases communicatively coupled to the patient status visualization system, and storing the retrieved historical physiological patient data in a memory of the patient status visualization system;
generating a patient status visualization of a status of the patient, based on the real-time physiological patient data;
displaying the patient status visualization on a display device; and
in response to a user adjusting a control element of the patient status visualization, retrieving one or more elements of the historical physiological patient data from the memory and displaying the one or more elements in the patient status visualization in real time as the user adjusts the control element.

2. The method of claim 1, wherein the control element may be adjusted by the user to a position within a range of positions, and the one or more elements of the historical physiological patient data retrieved from the memory correspond to a point in time indicated by the position.

3. The method of claim 2, wherein the control element is a slider.

4. The method of claim 2, wherein a chronological progression of the historical physiological patient data may be displayed by adjusting the position of the control element through the range of positions in a first direction, and a reverse chronological progression of the historical physiological patient data may be displayed by adjusting the position of the control element through the range of positions in a second, opposite direction.

5. The method of claim 4, wherein the chronological progression of the historical physiological patient data may be displayed by adjusting the position of the control element through the range of positions when the one or more systems and/or databases communicatively coupled to the patient status visualization system are inaccessible or in an unlaunched state.

6. The method of claim 2, further comprising:
in response to the user adjusting the control element to a position of the range of positions:
retrieving one or more elements of the historical physiological patient data from the memory corresponding to a time period between a current time and a time indicated by the position;
calculating an average of the retrieved one or more elements of the historical physiological patient data with the real-time physiological patient data;
and displaying the average in the patient status visualization.

7. The method of claim 1, further comprising:
continuously receiving the physiological patient data in real time from one or more patient monitors coupled to the one or more sensors, and
updating the patient status visualization in real time as the physiological patient data is received.

8. The method of claim 1, wherein the patient status visualization includes a radar chart visually depicting a status of the patient with respect to a plurality of patient status criteria categories of the physiological patient data, each criteria category including physiological patient data corresponding to a plurality of patient status criteria, the plurality of patient status criteria categories and the plurality of patient status criteria in each patient status criteria category of the plurality of patient status criteria categories configurable by the user.

9. The method of claim 8, wherein the plurality of patient status criteria categories further comprises:
a first category including physiological patient data corresponding to a circulatory system of the patient;
a second category including physiological patient data corresponding to a respiratory system of the patient;
a third category including physiological patient data corresponding to a balance of fluids of the patient; and
a fourth category including physiological patient data corresponding to an oxygen consumption of the patient.

10. The method of claim 8, wherein generating the patient status visualization of the status of the patient further comprises:
for each criteria category of the plurality of patient status criteria categories:
processing the physiological patient data of the criteria category to convert the physiological patient data into a single score for the criteria category;
placing a point at a distance from a center of the radar chart in a respective portion of the radar chart corresponding to the criteria category, the distance determined based on the single score; and
drawing lines between the points of the radar chart to form a network shape.

11. The method of claim 10, further comprising color-coding one or more points and/or lines of the radar chart based on the single score.

12. The method of claim 1, further comprising:
in response to the user selecting an element of the patient status visualization, displaying more detailed information of the patient status, the more detailed information including at least one of:
measurement values of physiological patient data of a criteria category;
a second radar chart depicting the physiological patient data of the criteria category;
a graph showing a trend in physiological patient data; and
a visualization of one or more physiological patient data superimposed on a depiction of a human body.

13. The method of claim 12, wherein the radar chart is configured to display a preview of historical physiological patient data in the patient status visualization; and
the more detailed information is displayed in response to the user directly selecting the preview, while the one or more systems and/or databases communicatively coupled to clinical information system are inaccessible or in an unlaunched state.

14. A patient status visualization system, comprising:
one or more processors, and a memory storing instructions executable by the one or more processors to:
generate a patient status visualization of a status of a patient of a healthcare system, based on physiological patient data received in real time from one or more patient monitors coupled to the patient;
retrieve historical physiological patient data corresponding to the physiological patient data from an electronic medical record (EMR) database of the healthcare system communicatively coupled to the patient status visualization system, and store the retrieved historical physiological patient data in a memory of the patient status visualization system;
display the patient status visualization on a display device; and
switch between displaying the real-time physiological patient data and the stored historical physiological patient data in response to a user adjusting a timeline slider element of the patient status visualization.

15. The patient status visualization system of claim 14, wherein the timeline slider element may be adjusted by the user to a position within a range of positions representing points in time, and historical physiological patient data corresponding to a point in time indicated by the position is displayed on the display device.

16. The patient status visualization system of claim 15, wherein further instructions are included in the memory that when executed, cause the one or more processors to:
in response to the user adjusting the timeline slider element:
retrieve elements of the stored historical physiological patient data occurring between the point in time and a current point in time;
display average data values of the elements of the stored historical physiological patient data on the display device.

17. The patient status visualization system of claim 14, wherein the patient status visualization includes a radar chart visually depicting the status of the patient with respect to a plurality of patient status criteria categories of the physiological patient data, each criteria category including physiological patient data corresponding to a plurality of patient status criteria, the plurality of patient status criteria categories and the plurality of patient status criteria in each patient status criteria category of the plurality of patient status criteria categories configurable by the user.

18. The patient status visualization system of claim 17, wherein further instructions are included in the memory that when executed, cause the one or more processors to:
for each criteria category of the plurality of patient status criteria categories:
process the physiological patient data of the patient status criteria category to convert the physiological patient data into a single score for the patient status criteria category;
place a point at a distance from a center of the radar chart in a respective portion of the radar chart corresponding to the patient status criteria category, the distance determined based on the single score; and
draw lines connecting the points of the radar chart to form a network shape that visually summarizes the patient status.

19. The patient status visualization system of claim 15, wherein further instructions are included in the memory that when executed, cause the one or more processors to:
in response to the user selecting an element of the patient status visualization, display more detailed information of the patient status, the more detailed information including at least one of:
measurement values of physiological patient data of a patient status criteria category;
a second radar chart depicting the physiological patient data of the patient status criteria category;
a graph showing a trend in a physiological patient data; and
a visualization of one or more physiological patient data superimposed on a depiction of a human body.

20. A method, comprising:
obtaining physiological patient data from one or more medical devices each monitoring a patient;
grouping the physiological patient data into four or more criteria categories relating to a status of the patient;
displaying, via a graphical user interface (GUI) displayed on a display screen, a first patient status summary that includes a metric for each criteria category of the four or more criteria categories derived from the physiological patient data; and
responsive to user input, displaying, via the GUI, a respective second patient status summary for each criteria category of the four or more criteria categories that includes a value for each element of the physiological patient data included in the category;
wherein the first patient status summary is a first radar chart that plots each metric in a different sector of the first radar chart with lines connecting each metric; and
each second patient status summary is a second radar chart that plots each value in a different sector of the second radar chart with lines connecting each value.
